(19) United States
(12) Patent Application Publication
(10) Pub. No.: US 2013/0085368 A1
(43) Pub. Date: Apr. 4, 2013

(54) Radiolucent ECG Electrode And Method Of Making Same

(71) Applicant: Scott R. Coggins, Littleton, MA (US)

(72) Inventor: Scott R. Coggins, Littleton, MA (US)

(73) Assignee: Tyco Healthcare Group LP, Mansfield, MA (US)

(21) Appl. No.: 13/249,541

(22) Filed: Sep. 30, 2011

(51) Int. Cl. A61B 5/0416; H05K 13/00

(52) U.S. Cl. 600/394; 29/458

(57) ABSTRACT

An electrode includes a pad having a patient contact side and a connector side. The patient contact side includes a conductive layer, and the connector side includes a press stud. An eyelet interconnects the conductive layer with the press stud. The eyelet includes a conductive coating disposed on at least a portion of a surface of the eyelet that contacts the conductive layer, while other surfaces of the eyelet are substantially free of the conductive coating.

### TECHNICAL FIELD

The present disclosure relates to biomedical electrodes, and in particular, to an ECG electrode having a radiolucent characteristic and method of making same.

### BACKGROUND

Electrocardiograph (ECG) monitors are widely used to obtain medical (i.e., biopotential) signals containing information indicative of the electrical activity associated with the heart and pulmonary system. To obtain medical signals, ECG electrodes are applied to the skin of a patient in various locations. The electrodes, after being positioned on the patient, connect to an ECG monitor by a set of ECG lead wires. The distal end of the ECG lead wire, or portion closest to the patient, may include a connector which is adapted to operably connect to the electrode to receive medical signals from the body. The proximal end of the ECG lead set is operably coupled to the ECG monitor and supplies the medical signals received from the body to the ECG monitor.

A typical ECG electrode may include a pad having an electrically conductive layer and a backing layer, the pad having a patient contact side and a connector side. The patient contact side of the pad may include a biocompatible conductive gel or adhesive for affixing the electrode to a patient's body for facilitating an appropriate electrical connection between the body and the electrode. The connector side of the pad may incorporate a metallic press stud or snap having a bulbous profile for coupling the electrode to the ECG lead wire. In use, the clinician removes a protective covering from the patient contact side of the pad to expose the gel or adhesive, affixes the electrode to the patient's body, and attaches the appropriate ECG lead wire connector to the press stud by pressing or “snapping” the lead wire connector onto the bulbous press stud to achieve mechanical and electrical coupling of the electrode and lead wire. After use, a clinician then removes the ECG lead wire connector from the pad by pulling or “unsnapping” the connector from the electrode.

An eyelet connects the electrically conductive layer of the electrode with the press stud to provide electrical communication therebetween. Currently available eyelets are either plated or completely coated with a conductive material. Some commercially available eyelets contain a large amount of silver, which may result in poor radiotransparency. Other products in the commercial market use a carbon filled plastic compound to improve radiolucency. However, these parts are also coated with silver material.

It would be advantageous to provide an ECG electrode having improved radiolucency and less silver than current commercially available electrodes.

### SUMMARY

The present disclosure is directed to an electrode having a radiotranslucent characteristic.

In one embodiment, an electrode comprises a pad including a patient contact side and a connector side, the patient contact side including a conductive layer and the connector side including a press stud; and an eyelet interconnecting the conductive layer with the press stud. The eyelet includes a conductive coating disposed on at least a portion of a surface thereof that contacts the conductive layer, wherein the conductive coating is present on the eyelet in an amount from about 15% of the surface area of the eyelet to about 50% of the surface area of the eyelet.

Another aspect of the disclosure is directed to a method of manufacturing an electrode comprising providing an eyelet, a conductive layer, and a press stud. A conductive coating is applied onto a distal surface of the eyelet. The electrode is assembled such that the distal surface of the eyelet contacts the conductive layer, and a proximal end of the eyelet contacts the press stud.

Other advantages, novel features, and objectives of the present disclosure will become apparent from the following detailed description of the present disclosure when considered in conjunction with the accompanying drawings, which are schematic and not intended to be drawn to scale. For purposes of clarity, not every component is labeled in every figure, nor is every component of each embodiment of the present disclosure shown where illustration is not necessary to allow those of ordinary skill in the art to understand the present disclosure.

### BRIEF DESCRIPTION OF THE DRAWINGS

Various embodiments of the presently disclosed ECG electrodes are disclosed herein with reference to the drawings, wherein:

FIG. 1 is a perspective view of an ECG electrode in accordance with an embodiment of the present disclosure;

FIG. 2 is a cross-sectional view taken along line 2-2 of FIG. 1; and

FIG. 3 is a cross-sectional view of an ECG electrode in accordance with another embodiment of the present disclosure.

### DETAILED DESCRIPTION OF EMBODIMENTS

Various exemplary embodiments of the present disclosure are discussed hereinbelow in terms of ECG electrodes for monitoring heart activity and for diagnosing heart abnormalities. It is envisioned, however, that the principles of the present disclosure are equally applicable to other medical electrodes, for example, electroencephalogram (EEG) electrodes; transcutaneous electrical nerve stimulation (TENS) electrodes used for pain management; neuromuscular stimulation (NMS) electrodes used for treating conditions such as scoliosis; muscle stimulation electrodes; wound treatment electrodes (accelerating healing of skin wounds or broken bones); defibrillation electrodes to dispense electrical energy to a chest cavity of a patient to defibrillate heart beats of the patient; iontophoresis; and dispersive electrodes to receive electrical energy dispensed into an incision made during electrosurgery.

In the following discussion, the terms “proximal” and “trailing” may be employed interchangeably, and should be understood as referring to the portion of a structure that is closer to a clinician during proper use. The terms “distal” and “leading” may also be employed interchangeably, and should be understood as referring to the portion of a structure that is further from the clinician during proper use. As used herein, the term “patient” should be understood as referring to a human subject or other animal, and the term “clinician” should be understood as referring to a doctor, nurse, or other care provider and may include support personnel.

“Radiotransparency” may be used interchangeably with “radiolucency”, and refers to the property of an electrode that allows a clinician to leave electrodes in place during radiological (e.g., x-ray) or other imaging examinations, to visualize the tissue underlying the electrode without loss of image quality.

The following discussion includes a description of embodiments of the presently disclosed ECG electrodes, as well as a description of exemplary corresponding methods of coating the same in accordance with the principles of the present disclosure.

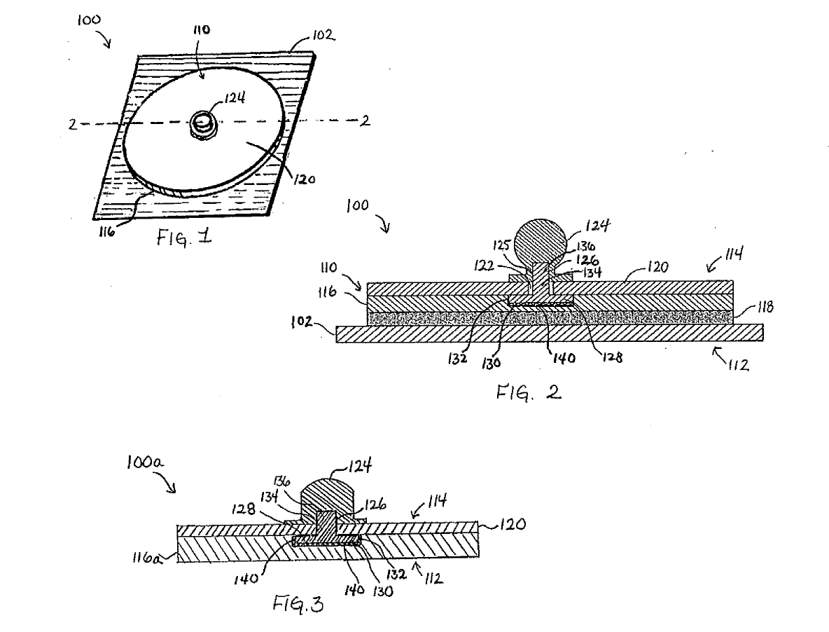

Referring now to the figures, where like components are designated by like reference numerals throughout the several view, FIGS. 1 and 2 illustrate an embodiment of an ECG electrode of the present disclosure. Electrode 100 includes a pad 110 including a patient contact side 112 and a connector side 114. The patient contact side 112 of pad 110 includes a conductive layer 116 and a conductive composition 118 for application to a body surface of a patient, e.g., a skin surface, for transmitting electrical signals and/or currents to and/or from the patient. Connector side 114 of pad 110 includes a non-conductive backing layer 120 having an opening 122 covered by a press stud 124 adapted for mechanical and electrical coupling with a lead wire (not shown).

The conductive composition 118 on the patient contact side 112 of pad 110 may be temporarily adhered to a release liner 102. Release liner 102 is a release paper or film of a waxed or coated plastic, such as a silicone coated polyethylene terephthalate film, which may be used to protect the patient contact side 112 of the electrode 100 prior to application of the electrode to the patient. The conductive composition 118 may be a conventional conductive gel. Other conductive compositions which may be utilized with the electrode 100 of the present disclosure includes hydrogels, such as, for example, those disclosed in commonly assigned U.S. Patent Application Publication Nos. 2009/0270709, entitled “Novel Electrodes”, and 2010/0059722, entitled “Conductive Compositions and Method”, the entire disclosures of each of which are hereby incorporated by reference herein.

FIG. 3 illustrates another embodiment of the presently described electrode shown generally as 100a. Electrode 100a is substantially similar to electrode 100, and will only be described as relates to the differences therebetween. In contrast to electrode 100, electrode 100a includes an adhesive conductive layer 116a, such as a solid hydrogel, for placement against the tissue of a patient.

As shown in FIGS. 2 and 3, eyelet 126 interconnects pad 110 with press stud 124. Eyelet 126 includes a base portion 128 positioned between the backing layer 120 and the conductive layer 116 (or 116a) of the electrode 100. The base portion 128 includes a distal surface 130 that is in contact with the conductive layer 116 (or 116a). Peripheral edges 132 of base portion 128 may also be in contact with the conductive layer 116 (or 116a) of the electrode 100. Eyelet 126 includes a post 134 extending from the base portion 128 in transverse relation to the pad 110 and protruding through opening 122 of backing layer 120. A proximal end 136 of post 134 is secured within a channel 125 of press stud 124, such as by friction fit or other conventional mechanical means.

The eyelet 126 may be fabricated of any suitable material. In embodiments, eyelet 126 may be fabricated from plastic. Non-limiting examples of suitable plastic materials from which the eyelet may be fabricated include polyolefins, such as polyethylene and polypropylene, including atactic, isotactic, syndiotactic, and blends and combinations thereof; polyethylene glycols; polyethylene oxides; ultra high molecular weight polyethylene; copolymers of polyethylene and polypropylene, as well as polyisobutylene and ethylene-alpha olefin copolymers; fluorinated polyolefins such as polytetrafluoroethylene and polyfluroroacetal; polyamides such as nylon and polycaprolactam; polyamines; polyimines; polyesters such as polyethylene terephthalate and polybutylene terephthalate; aliphatic polyesters; polyethers such as polyether ether ketone and polyether sulfonates; polyether-esters such as polybutester; polytetramethylene ether glycol; 1,4-butanediol; polyurethanes; acrylic polymers, copolymers, and resins; modacrylics; vinyl halide polymers and copolymers such as polyvinyl chloride; polyvinyl alcohols; polyvinyl ethers such as polyvinyl methyl ether; polyvinylidene halides such as polyvinylidene fluoride and polyvinylidene chloride; polyacrylonitrile; polyvinyl ketones; polyvinyl aromatics such as polystyrene; polyvinyl esters such as polyvinyl acetate; copolymers of vinyl monomers with each other and olefins such as etheylene-methyl methacrylate copolymers, acrylonitrile-styrene copolymers, ABS resins, and ethylene-vinyl acetate copolymers; alkyd resins; polycarhonates and alloys thereof; polyoxymethylenes; polyacetals; polyphosphazine; polysulfones; polymethylpentene; polyimides; epoxy resins; aramids; and combinations thereof.

In embodiments, the eyelet 126 may include a conductive filler material to enhance the flow of energy therethrough, Fillers include, for example, conductive metal fibers such as silver or tin fibers, and metallic threads, metallic powders, metallic flakes, and metallic spheres. The filler material may be carbon fillers, conductive carbon fiber fillers, acetylene black, chopped polyacrylonitrile fibers, noble metallic particles, noble metal halide particles, and combinations thereof. Suitable thermally conductive compounds include those commercially available, for example, from RTP Company.

In embodiments, the filler material may be admixed with, or impregnated within, the plastic material used to form the eyelet. The eyelet may be loaded with conductive filler material in amounts from about 2% by weight to about 75% by weight of the eyelet, in embodiments, from about 5% by weight to about 50% by weight of the eyelet, and in some embodiments, from about 10% by weight to about 40% by weight of the eyelet.

Alternatively, the plastic material forming the eyelet 126 may itself be conductive. Conductive polymers used to form such an eyelet include, for example, polythiophene, polyacetylene, polyphenylene vinylene, polypyrrole, polyaniline, polyphenylene sulfide, copolymers, and derivatives thereof, among other intrinsically conducting polymers within the purview of those skilled in the art. In embodiments, the conductive polymers may be utilized alone or in combination with conductive filler materials, as described above.

In embodiments, the outer surface of the eyelet 126 includes a topically applied conductive coating 140 that is adjacent to, and in contact with, the conductive layer 116 (or 116a) of the electrode 100 (or 100a). As illustrated in FIG. 2, the conductive coating 140 may be applied only to the base portion 128 of the eyelet 126, such that the rest of the eyelet 126 is substantially free of the conductive coating 140. In other embodiments, as shown in FIG. 3, the conductive coating 140 may be applied to the base portion 128 and peripheral edge 130 of the eyelet 126 (i.e., all the surfaces in contact with the conductive layer 116).

In embodiments, the conductive coating includes at least one heavy metal and/or heavy metal salt. Suitable heavy metals include, for example, the transition metals such as silver, gold, copper, zinc, cadmium, cobalt, nickel, palladium, and platinum, as well as some metalloids such as tin, bimetal and polymetal complexes, and salts thereof. In embodiments, the at least one heavy metal is silver or tin. Heavy metal salts include, for example, halide salts, such as fluorides, chlorides, bromides, iodides, and astatides of the above metals. Examples of heavy metal salts include, for example, silver salts such as silver acetate, silver carbonate, silver sulfate, silver phosphate, silver chloride, silver bromide, silver fluoride, silver citrate, and silver nitrate; and tin salts such as tin acetate, tin ethylhexanoate, tin sulfate, tin chloride, tin fluoride, tin iodide, tin bromide, and tin oxide, for example.

In embodiments, the heavy metal is coupled with a metal salt. In such embodiments, the metal/metal salt coating should have excellent conductivity. In embodiments, silver/silver chloride, tin/tin chloride, or other silver or tin metal/halide salt combinations may be utilized in the coating of the present disclosure. In some embodiments, a conductive coating containing a heavy metal may be applied to the eyelet and a metal salt complex may be formed by allowing a portion of the metal in the coating to convert into a halide salt by interaction with the conductive layer of the electrode.

In other embodiments, the conductive coating may be applied to the eyelet 126 prior to its use in manufacturing an electrode 100 or 100a. The conductive coating may be applied to the eyelet by any means within the purview of those skilled in the art including: spray coating; ultrasonic spray coating; electrospray coating; solvent/immersion coating such as dipping; solvent evaporation; combinations thereof, and the like. In embodiments, the metal and/or metal salt may be dissolved in any suitable solvent that is compatible with the base material forming the eyelet to accommodate the drying or curing time needed to deposit the coating on the eyelet. After application, the solvent may be evaporated, leaving the heavy metal and/or metal salt coating on the eyelet.

Alternatively, the coating may be applied to the eyelet by melt coating or electrostatic coating, among other techniques within the purview of those skilled in the art.

In embodiments, the conductive coating may include conductive filler materials, such as those described above.

In embodiments, the coating is an electrically conductive ink that may be transferred to the surface of the eyelet by any of a number of transfer, printing, or laminating methods, such as screen printing, pad printing, or hot stamping. Suitable inks, such as silver/silver chloride inks, are commercially available from Ercon Inc. (as ERCON R68 ink) and Henkel Corporation (as ELECTRODAG PE-007 ink).

In embodiments, the conductive coating may be present in an amount from about 1% by weight to about 5% by weight of the eyelet. In one embodiment the conductive coating may be present in an amount of about 2% by weight of the eyelet.

In embodiments, only a portion of the surface of the eyelet may possess a conductive coating thereon. The remaining surface of the eyelet may be substantially free of the conductive composition. For example, in embodiments the conductive composition may be present on the eyelet in an amount from about 15% of the surface area of the eyelet to about 50% of the surface area of the eyelet, in embodiments from about 20% of the surface area of the eyelet to about 45% of the surface area of the eyelet. The conductive coating may by silver chloride with a silver content of about 15% to about 18% silver by weight.

For example, an eyelet may be formed from about a 30% to about a 40% carbon filled plastic. A silver/silver chloride ink is pad printed onto the distal surface of the eyelet. The electrode is then assembled such that the distal surface of the eyelet containing the silver/silver chloride ink contacts the conductive layer of the electrode, and the proximal end of the eyelet, which is free of a conductive coating, contacts the press stud.

In use, the electrodes of the present disclosure have excellent conductivity. In addition, as the electrodes of the present disclosure do not possess eyelets completely coated with a conductive material, such as silver, they have excellent radiotransparency, i.e., radiolucency.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, the present disclosure is not intended to be limited to the details shown, since it will be understood that various omissions, modifications, substitutions, and changes in the forms and details of the device illustrated and its operation can be made by those skilled in the art without departing in any way from the spirit and scope of the appended claims.

What is claimed is:

1. An electrode comprising:
a pad including a patient contact side and a connector side, the patient contact side including a conductive layer and the connector side including a press stud; and
an eyelet interconnecting the conductive layer with the press stud, the eyelet including a conductive coating disposed on at least a portion of a surface thereof that contacts the conductive layer,
wherein the conductive coating is present on the eyelet in an amount from about 15% of the surface area of the eyelet to about 50% of the surface area of the eyelet.

2. The electrode of claim 1, wherein the eyelet is formed from plastic.

3. The electrode of claim 1, wherein the eyelet includes a conductive filler material.

4. The electrode of claim 3, wherein the conductive filler material is selected from the group consisting of metal fibers, metallic threads, metallic powders, metallic flakes, and metallic spheres.

5. The electrode of claim 3, wherein the conductive filler material is selected from the group consisting of carbon fillers, conductive carbon fiber fillers, acetylene black, chopped polyacrylonitrile fibers, noble metallic particles, noble metal halide particles, and combinations thereof.

6. The electrode of claim 3, wherein the eyelet contains from about 2% by weight to about 75% by weight of the conductive filler.

7. The electrode of claim 3, wherein the eyelet contains from about 5% by weight to about 50% by weight of the conductive filler.

8. The electrode of claim 2, wherein the plastic comprises a conductive plastic selected from the group consisting of polythiophene, polyacetylene, polyphenylene vinylene, polypyrrole, polyaniline, polyphenylene sulfide, copolymers, and derivatives thereof.

9. The electrode of claim 1, wherein the eyelet includes a base portion and a post.

10. The electrode of claim 9, wherein the base portion includes a distal surface including the conductive coating and the post is substantially free of the conductive coating.

11. The electrode of 10, wherein the base portion further includes a peripheral edge including the conductive coating.

12. The electrode of claim 1, wherein the conductive coating includes a heavy metal.

13. The electrode of claim 12, wherein the heavy metal is selected from the group consisting of silver, gold, copper, zinc, cadmium, cobalt, nickel, palladium, platinum, tin, bimetal and polymetal complexes, salts thereof, and combinations thereof.

14. The electrode of claim 12, wherein the conductive coating includes a conductive filler material.

15. The electrode of claim 1, wherein the conductive coating comprises an electrically conductive ink.

16. The electrode of claim 15, wherein the electrically conductive ink comprises a silver/silver chloride ink.

17. A method of manufacturing an electrode comprising:
providing an eyelet, a conductive layer, and a press stud;
applying a conductive coating onto a distal surface of the eyelet; and
assembling the electrode such that the distal surface of the eyelet contacts the conductive layer, and a proximal end of the eyelet contacts the press stud.

18. The method of claim 17, wherein the conductive coating comprises an electrically conductive ink applied by one of printing, laminating, or stamping.
